(19) United States
(12) Patent Application Publication
(10) Pub. No.: US 2020/0243194 A1
(43) Pub. Date: Jul. 30, 2020

(54) Computerized Medical Diagnostic and Treatment Guidance

(71) Applicant: Joseph Alan Epstein, Pleasanton, CA (US)

(72) Inventor: Joseph Alan Epstein, Pleasanton, CA (US)

(21) Appl. No.: 16/751,069

(22) Filed: Jan. 23, 2020

Related U.S. Application Data
(60) Provisional application No. 62/797,262, filed on Jan. 26, 2019.

(51) Int. Cl. G16H 50/20; G16H 50/70; G16H 10/60; G16H 10/40; G16H 50/50; G16H 50/30; G16H 70/60; G16H 70/20; G16H 80/00; G16H 15/00; A61B 5/00

(52) U.S. Cl. CPC G16H 50/20; G16H 50/70; G16H 10/60; G16H 10/40; G16H 50/50; A61B 5/021; G16H 70/60; G16H 70/20; G16H 80/00; G16H 15/00; A61B 5/7275; G16H 50/30

(57) ABSTRACT

A method and system for aiding in the diagnosis and treatment of medical and health conditions. In some embodiments, a risk profiler analyzes population incidence statistics with test results to populate an interactive simulation and decision engine. Further embodiments produce treatment betting tables. In some embodiments, diagnosis fact boxes are produced. Users, such as clinicians and patients, interact with the simulation and decision engine to explore different diagnosis possibilities and treatment options, with feedback showing the statistical likelihoods and consequences of the possibilities.

### CROSS REFERENCE TO RELATED APPLICATION

This application claims the benefit of provisional patent application Ser. No. 62/797,262, filed Jan. 26, 2019 by the present inventor, the entire content of which is hereby incorporated by reference.

### BACKGROUND OF THE INVENTION

### 1. Field of the Invention

The present invention relates to the field of electronic tools to diagnose or assist in the diagnosis of medical and health-related conditions, including population statistics, cohort analysis, displays for the user—some of which may be interactive—and medical statistical information representation and presentation to aid in the understanding of a diagnosis.

### 2. Description of the Prior Art

The cost and complexity of medical care has been exploding. One main reason for this is that the primary care physician is unable to adequately treat the patient. With the limited time the PCP has with the patient on each encounter—15 minutes, typically—the PCP cannot learn enough about the patient to be a competent advocate for his or her health. Each patient's body responds differently to treatment, to experiences, to life itself, and the PCP finds herself unable to remember enough details about the patient to adequately address the patient's current condition. The electronic health record system is of no help, as it only lists the obvious chronic conditions that the patient could have explained in one minute. Details that truly would light the way to treatment are buried within a running stream-of-consciousness narrative of past encounters, themselves made of copy-and-pasted notes from even earlier encounters.

Furthermore, doctors are simply unable to be fully aware of or remember all of the latest research, or even the methods of care, for most conditions. PCPs find themselves being forced to recommend patients off to specialists almost immediately for any condition, given that they themselves are not the expert for the specialty, and are unable to know or access enough knowledge to treat the patient without the risk of committing a grievous error for missing some point that the specialist would never do, given that the specialist sees these conditions every day but the PCP only occasionally. Something as basic as a nose fracture can easily get misunderstood. The PCP may correctly order a facial X-ray, which a separate radiologist will read. This may properly rule out a cheek or orbital fracture or bone fragments broken loose. But the PCP may not remember that an X-ray is not dispositive for nasal fractures, and that the standard of care is not to X-ray but to palpate, as only feeling the position of the nasal bones will show if they are in the proper position. The X-ray will not be a sensitive enough a test, as the nasal bones will be occluded, and will have tremendous false negatives.

On top of that, the availability of genetic and epigenetic information on the patient, coupled with extensive but inchoate research into which genetic markers can serve as indicators of increased risk of a condition and by what amount, demands that the physician take into account each patient's likely response to their environment and the treatment. Physicians, of course, do not have enough time in the day to read and understand all of that research, and if they did, that could actually be dangerous given that the research is constantly shifting. That doesn't mean that the information is garbage, but rather that it hasn't reached the level to be incorporated into the standard of care for every patient. Yet the information within that research could be tremendously valuable—a genetic predisposition to diabetes should cause a physician to become particularly concerned about a patient's obesity, whereas the opposite should allow confidence to wait until standard blood indicators (AlC, say) show that diabetes is setting in.

Finally, physicians, like all people, are not particularly good with statistics. Professors of small classes grade on a curve, not remembering the obvious fact that the sample size is too small, and furthermore not seeing that the population covaries, as a group of intelligent students might all sign up for the same class for convenience—especially if it has partner or lab work. Closer to this subject, physicians will strongly suggest that someone take a test with a 5% false positive rate to be sure they don't have a life-altering disease, even if the incidence rate of the disease itself is far less than 5%, making false positives the most likely positives. This matters because the modern practice of medicine must be based on statistics. Genetic and epigenetic tests provide multiples of increased risk, yet on low incidence diseases. A 250% increased risk of developing pancreatic cancer, say, is not something that can be interpreted properly without understanding and accessing enough surrounding context. What is the baseline risk, and the rate of incidence? If this is a lifetime risk, how does the risk get properly broken down into age bands? When should a physician start to test? When should a physician start taking the tests more seriously? The answers are all statistical in nature—but, as experts such as Daniel Kahneman have taught us, people are not good at performing statistical analysis in their heads.

But computers can be.

Today's state of the art is shockingly poor in this area. In large part, this is because the problems mentioned above are not issues of records, but of analysis and professional knowledge as it relates to it. There are a large number of specific diagnostic tools, from automated radiology reading to genetics counseling packages. But these are very specialty and disease specific. There, to date, has been no effective and widely embraced system for providing the guidance.

The current paradigm is for the EHR to provide the physician with access to as much patient-specific data as possible. Separately, a physician is free to pick up or electronically access a diagnostics manual—but, as mentioned before, they won't have the time in the encounter to do this, and the manual itself is likely to be woefully inadequate. It really doesn't make sense for an EHR system to be the primary tool a physician uses, because EHRs are primarily, as the acronym Electronic Health Records suggest, records of past state and not tools for diagnostics. There have been some attempts to put the simplest, most brain-dumb assistance into the EHR. A physician ordering a PSA test will now possibly be presented with the equivalent of a box warning—not patient specific, and retrofitted into the ordering system of the EHR—that PSA tests have a false positive rate high enough to suggest that they ought not to be ordered in many cases. But how does it apply specifically to this patient—where the EHR has some information about the patient's family history, and where genetic or patient-specific testing kept outside of the EHR would greatly improve the prediction in this case? Tools today cannot tell a doctor what conditions are really more likely right now—based on the actual data of the patient and not just on population trends and how certain indications present—and what can be pushed to the background. Some tools today try to tell a doctor what to do—or strive (and fail) to replace physicians under the false hope that a computer is all one needs—but they are not created to act as a physician's friend, something that makes the physician far smarter and wiser because of the expansive depth of knowledge and calculation the electronic tool can perform.

### SUMMARY

In accordance with some embodiments, a method and system for making informed medical decisions about a patient, based off of a simulation and decision engine driven by a risk profiler populated with population incidence statistics and patient test results, diagnoses, and other relevant data. Interactive displays show the users the statistical relevance of information and allow the user or users to explore the likelihoods of diagnoses assumptions, treatments, and further testing, along with their comparisons.

### BRIEF DESCRIPTION OF THE DRAWINGS

FIG. 1 is a diagram of an embodiment of the invention, showing a risk profiler communicating with a simulation and decision engine, along with various sources of data.

FIG. 2 is a diagram of an embodiment of the propensities and incidences system.

FIG. 3 is a diagram of digester tables such as produced by and consumed by embodiments of this invention.

FIG. 4 is a diagram of an embodiment of this invention, producing a patient path.

FIG. 5 is a diagram of a simple patient path such as those produced by and consumed by embodiments of this invention.

FIG. 6 is a diagram of a more complex patient path than that in FIG. 5.

FIG. 7 is a diagram of an embodiment of this invention, producing the contents of an interactive display based on analysis, prediction, and simulation.

FIG. 8 is a diagram of the interactive display of an embodiment of this invention.

FIG. 9 is a diagram of one type of visual expression of a betting table for the analysis of the probability of resolution such as produced by an embodiment of this invention.

FIG. 10 is a diagram of one type of visual expression of a betting table for the total risk to the patient such as produced by an embodiment of this invention.

FIG. 11 is a diagram of one type of visual expression of multiple betting tables, representing different treatment paths, such as produced by an embodiment of this invention.

FIG. 12 is a diagram of an embodiment of this invention, showing a decision state space explorer and chart navigator producing betting charts based on user input as a part of simulation.

FIG. 13 is a diagram of an embodiment of this invention, showing collaboration mechanisms between multiple users operating their own simulation and decision engines, with joint revision control and commenting.

FIG. 14 is a diagram of a fact box as produced by an embodiment of this invention, highlighting diagnostic statistics in a way to overcome inherent misconceptions and biases of the observer of the fact box.

FIG. 15 is a diagram of an embodiment of this invention, showing the integration of the user interface of this invention into an EMR/EHR system.

### DETAILED DESCRIPTION

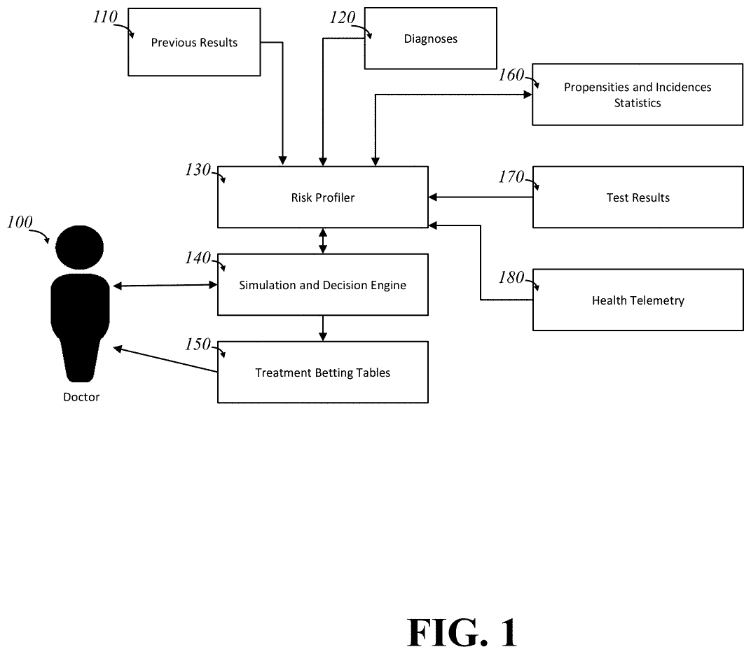

Some embodiments of the invention produce a probability weighting dashboard for medical decision making based on the specific user data. FIG. 1 shows the architecture of some embodiments of the present invention. The embodiments collect at least one of health telemetry 180—such as a stream of at-home blood pressure, EKG data, and other health data collected, say, from wearable devices—as well as test results 170—such as clinically performed diagnostic tests and lab tests—and a queriable system 160 containing population and subpopulation statistics—such as incidence rates, comorbidities, correlations, and causalities—and previous diagnoses 120 and previous results 110 from the embodiments. A risk profiler 130 gathers the information and uses it to create or update a model of the patient, containing his potential conditions and how they relate to past or future events. One such embodiment uses Bayesian statistics to maintain prior and posterior records of the statistics for the conditions being tracked. Another embodiment maintains likelihoods for known patient state transitions—basically, what statistics would need to be adjusted and how if the patient's state were to transition from one state description to another, along with the likelihoods determining that state. A further embodiment maintains at least subsets of statistical distributions for the range of conditions and states of a patient, as opposed to merely a discrete state-probability pair.

The risk profiler feeds a simulation and decision engine 140. This engine simulates the patient's likely response to any number of external events, including potential treatments and results of new tests. The point of the engine is to drive the ability for a clinician to “play around” with a medical avatar, as it were, of the patient. The invention helps answer questions like “What could happen if we give the patient this intervention?” or “How much more confident can we be if we performed the following additional test?” The doctor is able to inform the system of the what-ifs and watch the scenario analysis unfold. In one embodiment, the updates and results are produced in real or near real time.

In some embodiments, treatment betting charts or tables 150 are produced. Betting charts are used broadly here to mean visual, tabular, or electronic displays of what the physician and system together or separately (in one or many different “avatars” of the patient) of what diagnoses are possible and what different interventions (including diagnostic tests) could do. In some embodiments, the clinician can go and temporarily or permanently delete or alter past assumptions, to derive at for example what an average person in the same patient cohort would be subject to, and to even show the difference.

Population incidence data can be quite varied and conditional, populated using epidemiology, but fine grained. The key to understanding the right incidence rate that can apply to a patient is to get close to correctly placing her into a cohort, and then to also correctly interpret the facts leading to the suspicion. The consequences of producing the wrong cohort can be severe. For example, some studies show the background incidence rate for moderate obstructive sleep apnea (15 to 30 Apnea-Hypopnea Incidences or AHIs per hour) is 1 in 15, or almost 7%. However, other studies have shown the rate being higher for men and lower for women: almost 2:1. Furthermore, studies have shown that a subsequent 10% increase in weight produces a 32% increase in the possible AHI, and therefore risk of moderate or severe OSA. A man with a recent history of weight gain might properly have an 11% (male) incidence rate for having moderate sleep apnea, but with the AHI multiplied by 132% for the weight, as assuming uniform distribution within the 15-30 AHI now shifted to 20-40 AHI, the incidence rate of severe OSA could now be 11%×½ (half of the cohort now has AHI over 30, the severe cutoff), or 6% added severe on top of the background rate.

Unfortunately, there are more studies than a person can read. And usually only sleep physicians, to continue the above example, would read those articles or even know of their existence. But this invention can help.

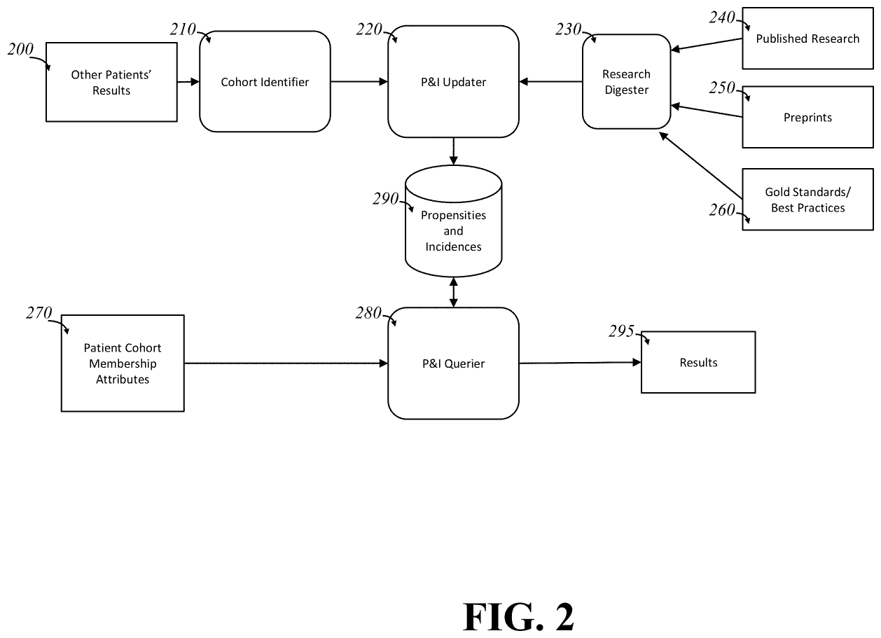

FIG. 2 shows the method of producing and matching cohort propensities and incidence rates in some embodiments of the present invention to produce queryable propensities and incidences (P&I) statistics 160. In these embodiments, cohort information and statistical knowledge is assembled from at least one of the following. Published articles 240 and prepublished articles 250 showing research with relevant epidemiological and statistical information are gathered by a Research Digester 230, along with optional existing best practices and standards manuals 260. In some embodiments, the Research Digester produces this information manually, through one or more people who, when given the article in question, extract out the defining cohort membership attributes from the study (such as sex, age group, life events, family history) as well as the propensity and risk information, such as the various adjustments to likelihoods or raw incidence rates as seen. For example, if the study in question shows that across a panel of unspecified age and gender, losing 10% of body weight was shown to produce a 26% decrease in likely AHI, such information is entered in to be passed to the P&I Updater 220 tied with the update. One specific embodiment allows for the entry of said cohort panel attributes and results in tabular form—a digester table—as if in a two-column, two table spreadsheet where the left column of table one contains rows where the inputer selects for each row the specific attribute type (such as Gender, Weight, etc.), and the right column contains the range or specific defining characteristics. The second table's left column includes the relevant diagnostic property, and the right column shows the adjustment. A further embodiment allows choosing the types from a known prepopulated list. A further embodiment allows entering ranges and adjustments in computer-parseable symbols. Note that the results need not be stored in tables: that is merely the procedure in some embodiments.

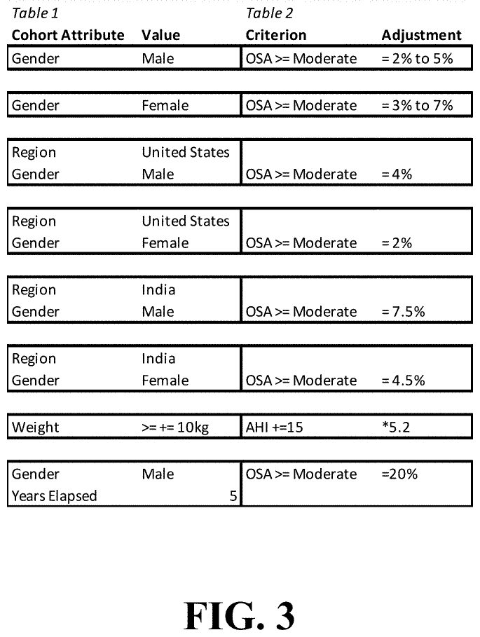

One such sets of digester tables produced by an embodiment of this invention is presented in FIG. 3. The Table 1 side (left) shows the population attributes that must match: the Table 2 side (right) shows the adjustments to the incidence rate or diagnostic criteria. This particular table was encoded, without loss of generality, using a declarative language: some embodiments parse a language for the entries to determine the meaning of the particular entries. For example, the fourth box shows the Region to be the United States and the Gender to be Female, thus matching American women. The resulting adjustments show that the diagnosis for OSA (obstructive sleep apnea) at least (>=) Moderate has a declared (=) incidence rate of 2%. The second to last box shows a variation: if the weight change (over no specified amount of time) is over 10 kg, then the AHI of +=15 (meaning that AHI increases by 15) has a probability increase of 5.2. In the case of the above table, each row in Table 1 functions as a logical AND; each row in Table 2 functions as its own consequence. The declarations may be processed for logical consistency and circular reasoning; in one embodiment, such errors are flagged for the user who is inputting in real time: a more specific embodiment shows the errors in the manner of a spreadsheet, with arrows showing dependencies and errors. The table above uses a specific language, but other languages are also usable, including functional languages, text-only languages, and natural languages: using natural language processing is an embodiment of the invention.

Some other embodiments generate the digested information automatically. Some such embodiments use natural language processing on top of the entire document, with the effect of scanning the document and determining where the important information is. One such embodiment trains machine-learning NLP algorithms on medical research. A further embodiment uses supervised training at least in part, where the appropriate results (such as a known or manually produced digestion table) is used to establish the intended results. Another embodiment uses humans to double check the work independent of the learning style: a specific embodiment uses confidence information produced by the AI processor to place ambiguous or low confidence results in front of humans for double checking. Another embodiment uses statistical sampling models and processes to identify a subset of results to check with humans to improve the overall statistical confidence of the batch. It is optional, but often desirable, to attach to the digested information the specific source article, line, version, and similar from where the data was collected. The outputs of the digestion (digestion tables in some embodiments) is entered into a Propensity and Incidence (P&I) Updater 220.

A second input is from actual practice data. In some embodiments, the statistics of the existing practice 220 are derived, either manually or automatically, and inserted into the P&I Updater 220 with cohort identification 210 (either unique or fuzzy, as the P&I block can handle fuzzy data). In one embodiment, the data is also entered into digester tables. In some embodiments, a patient is categorized into his cohort and then further information is produced that shows that the cohort classification may have been wrong, this information is introduced into the P&I Updater 220. Some embodiments of the cohort identifier merely produce a matching patient cohort membership attribute list and have the P&I updater 220 apply similar access patterns as the querier.

The P&I Updater 220 introduces the new data into the P&I element 290. There are many different embodiments of the P&I element 290. One thing to notice is that the inputs into the cohort learnings may be fuzzy, or even quite contradictory. In some embodiments, the cohort system is configured to produce a reasonable syncretic state of the knowledge at least to the view of the P&I Querier 280. This means synthesizing or reconciling conflicting data. For example, one study may set a baseline rate for a population group at one number; another may have it at a different number. Or a study may show the variations for some groups but not fully specify the data: if a study says that weight gain produces an increase in the risk of something, but doesn't specify over what time the risk increases, there is a potential for ambiguity or conflict. There are many systems known to the art that can handle this sort of data. A non-machine-learning construct embodiment employs an appending-only database of the various declarations of consequences, and provides the entire set of matching consequences upon an appropriate cohort attribute query (a pattern matching database is one such implementation), thus requiring the querier to determine what to do with the information. Some embodiments use machine learning. In one embodiment, a neural network is constructed, with new rules inputted as new training data. A further embodiment specifically uses query attributes and the multiple results from the above mentioned pattern matching database and backpropagates each of the conflicting results, thus letting the nature of the particular neural network synthesize the differences as per the art. Similar embodiments apply the updates into constructing Markov models.

The P&I element 290 may and will usually contain stable storage, such as a database or neural network training repository, for the purpose of persistence. In some embodiments, the continuous operation and updating of the P&I element 290 such as through the disclosed mechanisms may tend to improve the output content of the P&I system as figured. As the P&I element 290 therefore develops more “knowledge”, the value of the P&I element 290's outputs may go up. At scale, such a P&I system may have value in and of itself as a global repository of unified propensities and incidence rates.

Queries come in through the lower blocks in the figure. A query may be constructed in multiple ways in some embodiments. The figure shows one such query, constructed by drawing out (strict or fuzzy) attributes of a patient 270—real or hypothetical. These attributes are then sent into the P&I Querier 280. In some embodiments, the P&I Querier takes the same inputs as Table One of the above-mentioned digester table. Other embodiments take queries based on a query language as mentioned above. The P&I Querier 280 consults the P&I element 290 and produces the requisite results 295. In one embodiment, the results 295 are a matching set of digester table entries showing propensity relationships: these may be a harmonized Table 2 only matching the query request, or it may include all of the subqualifications based on further detailed query possibilities not necessarily provided by the query itself. Some other embodiments return results matching the established state of understood propensities.

When using a neural network with inputs corresponding to the set of cohort attributes (such as decomposed or coded ranges, procedural inputs, often implemented as categorical coding reductions, preprocessing convolutions, and other input-to-neuron mappings that are available in the art) and outputs corresponding to known propensity rules (again mapped as known to the art), the outputs for each specific instantiation of a fully developed rule (say a rule for “AHI>=15”), some embodiments retain the use of a harness to develop and remember new instantiations of inputs and outputs and new nodes added based on further rule development. In one embodiment, the harness is reviewed by a supervisor (human or itself an automatic system focused on rule coherence, such as a neural network trained by nested or overlapping rules by backpropagating logical relations between the outputs to allow the detection of inconsistent or rule-violating outputs [probabilities greater than 100% for example]) to ensure that the underlying network is consistent. In one embodiment, the outputs correspond to coded rules such that each output neuron's output represents or correlates to the confidence of the network in that the output consequences matter.

Overlapping algebraic or logical meaning in inputs and outputs are handled by some embodiments. For example, picture a network trained with two outputs corresponding to two different left columns of a Table 2 digester table, say “AHI>=15” and “AHI” itself. These are related. If the network can output a prediction for AHI of 20, as well as a probability that the AHI>=15, then the results do need to be correlated for sense to be made. The P&I Updater 220 and the P&I Querier 280, in some embodiments, share joint logic for P&I reconciliation. In embodiments where the inputs and the outputs are parsed or otherwise encoded into a language and the language itself can be represented in a structure where relationships are deducible, the employment of a language reconciliation engine with the inputs in some embodiments create a suitable input or output coding. In the AHI case, whenever the P&I Updater 220 in these embodiments trains on a specific AHI number, all related AHI relations that have other outputs that can be deduced are also set appropriately. It is possible that in some cases the trainings will result in inputs that can lead to the network overfitting the distinctions and producing an inconsistency such as the AHI number neurons being output with, say, 20 at high confidence but the AHI>=15 line being left off or at low confidence. In those cases, the same language reconciliation engine can force consistency in the output by recalculating AHI>=15 and other dependent outputs as needed as a matter of course. Note above that that was also used to train, but sometimes trainings may not stick properly. Also note that specific trainings can be provided almost entirely automatically by finding some or all logically related input codes (say Weight>100 kg and Weight>75 kg) and finding activations of outputs where there are logical inconsistencies and reasserting the consistent output for training. Although automated machine learning codings are not always predictable, it is expected to be likely that the automated input codings generated are coded so that a signal is asserted in the input neuron corresponding to an appropriate input rule, and the absence of a signal is the absence of assertion.

Techniques for machine learning harnesses such as described in application Ser. No. 16/293,422 are applied here in some embodiments, thus possibly allowing for multiple competing networks to be automatically supervised and some rejected as needed. This can be useful when some arena candidate networks have greater logical consistency than others.

P&I learning has so far been described based on population attributes in general, and used examples that are specific to the state or trait of the patient. Those are not the only attributes available. The diagnostic or testing path may also be added into the inputs. So may test results and telemetry. This must be done with some care, because most machine learning techniques are not Bayesian and may lead to unusual overfitting. However, it is clear that the P&I system's input classification has no idea whether the inputs are states, traits, or arbitrary labels and relations, and thus these can be coded. Some embodiments do perform this added information delivery. However, below is disclosed an alternative method for handling diagnostic path.

In the embodiments where the P&I system 160 takes in mostly or only just incidence statistics, the Risk Profiler 130 takes the role of combining the remaining information for presentation to the Simulation and Decision Engine 140. Different connections provide possibly different benefits, allowing the choice of embodiment to fit the needs of the particular instance and use.

In many embodiments the risk profiler 130 produces, as expected, a risk profile. A risk profile is a coherent set of risks based on the assumptions it is fed. These assumptions include information that can be used to determine which cohort the patient is in (though note well that neural network and other machine learning embodiments of the P&I system may not have strictly determinable cohort sets but instead are probably finding some sort of topological relationships between inputs in a less structured way). In these embodiments, it is in the risk profiler where the non-cohort patient path is taken into account. Please note that removing the risk profiler and feeding its inputs into the P&I system 160 is an embodiment of the invention, but this section will proceed to describe the embodiments with a separate Risk Profiler 130.

Some embodiments use a patient path. A patient path is the set of encounters and decisions that got the patient to this point. It may contain highly relevant information in a proper Bayesian analysis (or other similar adaptive predictive analyses) of the patient's situation. Patient paths can be thought of as extending both into the past and the future, and in fact are often not entirely known or knowable. More specifically, the current state of the patient and thus the current path may be fuzzy or incompletely determined. However, depending on the amount of data that the employer of the invention wishes to capture, it may be possible to find more certain points in the state space of the patient and determine what limitations exist to the potential patient state. (An analogy worth thinking about is in indeterminacy in quantum mechanics, where one ought to calculate all possible paths the particles in a system may take—or at least do so between points of “collapse” or determinacy.) This is one of the potential advantages of this risk profiling: it does not require an absolute determination of the diagnoses or conditions of the patient, but instead can handle the indeterminacy in a responsible manner, properly narrowing possibilities as the evidence allows. This creates an opportunity for true end-to-end evidence-based medicine, even though the evidence may be inconclusive in points. Such information is very difficult for an individual person to understand at any one time. The reason for having patient path information is to help manage prior and posterior inferences for each encounter, and to—as will be described later—allow a physician the discretion to take into account or discount parts of the path and related diagnoses to determine what possible risk the patient is in based on her medical judgement, thus not requiring perfect accuracy of the electronic system and allowing for the human art of medicine to shine.

Why the patient path matters is precisely in assessing how much to trust the prior probabilities. This is an intrinsically natural problem, especially in the medical specialties. Most patients present to a specialist with something that strikes close to the purview of the specialist. But not all. And this leads to a strange but real structural problem with specialists or even specific diagnoses, which is that treatments can have high outcome percentages in the specific and yet be worse than a guess. The reason for this is fairly simple: patients self-refer or are referred to specialists most often because they believe they are good candidates for the services offered, thus greatly increasing the prior probabilities. However, the subgroup of patients who get referred who are not good candidates retain their low prior probability, and yet get lumped in.

This is endemic with expertise for special situations in general: because special situations are rare, and yet most tests and treatments have statistical specificity in the 90-95% range, most expert opinions stand an excellent chance of being wrong if applied to a random member of the populace. This leads to an interesting—and famous—blend of confirmation bias and general blindness, and is why general screening is often a terrible idea for most tests known in practice settings to be highly effective (and a 95% specificity test is quite effective). If police officers gave breathalyzer tests to random drivers, they would end up arresting more innocent than guilty drivers, even though a breathalyzer test has excellent specificity, simply because 1—specificity rivals the incidence rate. (This is a rule of thumb, and the full math requires the sensitivity too, but the point is the same.)

This vexing problem of split distributions, where so many are properly treated and then a few who are in the wrong place at the wrong time get misdiagnosed to some degree or another. A specific example will help, so let's return to the realm of obstructive sleep apnea. Most people who go to a sleep physician do so because they cannot sleep well, and often because their spouse or bedmate has informed them that they snore heavily, toss often, or sometimes seem to stop breathing. However, a small segment gets referred by other physicians, say primary care and cardiology, when trying to understand idiopathic high blood pressure. The first group have already fit so many criteria that are consistent with, even if not dispositive for, sleep apnea that their prior probabilities should be adjusted well upwards. And it is clear at this point to see that a Headache Clinic like model of treating OSA almost blindly would be profoundly successful. Except for the people referred for idiopathic hypertension. The probability of hypertension given sleep apnea is fairly high. The converse probability is fairly low, though application of Bayes' theorem. Therefore, for this population set, the value of the diagnostic tests and treatment possibilities must be carefully monitored. OSA at home tests have around 80% sensitivity and specificity for moderate OSA. For people with high prior probability of OSA, this test will be very useful. Yet the background incidence rate for it is, say, 6% for a random patient. The probability of having OSA merely given hypertension is fairly low, perhaps no higher than 8%. Unfortunately, the positive predictive value (PPV) of the at home OSA test calculates to around 25%, meaning that given a positive result, 75% of the patients with nonelevated priors do not have OSA. Physicians, however, are often unaware of this. It is not uncommon for physicians to regard the false positive rate as being the probability of the test being wrong given a positive result, even when it really means the ratio of false positives to actual negatives, a far smaller number for rare diseases.

The risk profiler 130, in these embodiments, is charged with processing the patient path, gathered through the diagnoses 120 and test results 170, previous rounds of processing of this system 110, health telemetry 180, population propensity and incidence statistics 160, and additional assumptions or relaxations of criteria presented by the Simulation and Decision Engine 140 to produce the risk profile. The patient path is derived from the past diagnoses, tests, and conditions enforced by the simulation engine to establish the chain of prior and posterior probabilities.

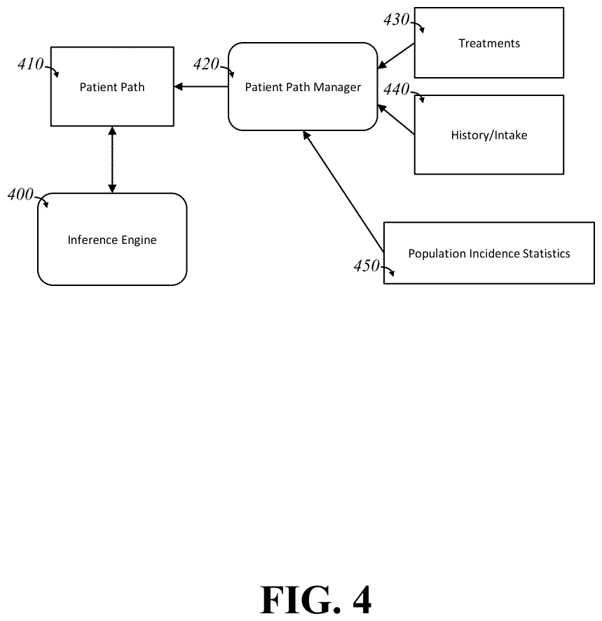

FIG. 4 shows the patient path production in some embodiments. A Patient Path Manager 420 is charged with assembling the data of a patient path 410 from sources such as the history of treatments 430, the intake patient history 440, and queries from the P&I system 450. This produces a known history of the patient including all interventions. These interventions are then assembled into a timeline: a patient path 410. Once the timeline (or a partial subset) is produced, an Inference Engine 440 acts upon the timeline to produce inferences. An inference is an assessment for a patient path of the propensity for a patient to fit a particular belief or diagnosis. As disclosed below, there are a number of embodiments that fulfill this requirement. One example is to use Bayesian inference, starting with the cohort incidence rate and applying likelihood updates to that rate based on the particular steps along the timeline. But, of course, other forms of inference are available, such as maximum indifference.

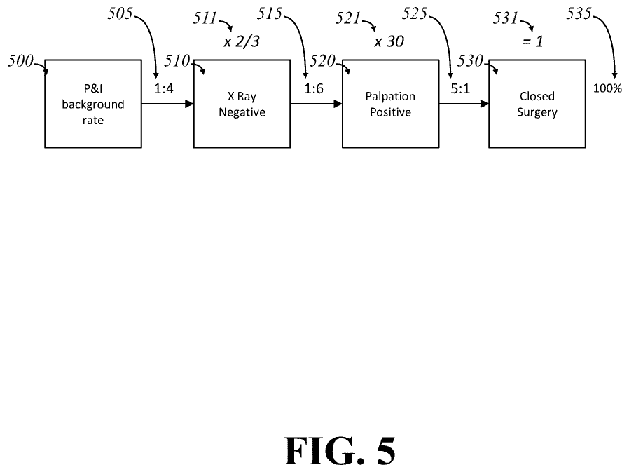

FIG. 5 shows an example patient path for a broken nose in some embodiments. The probabilities 505, 515, 525, and 535 have been shown as odds ratios instead, to show the operation of likelihood adjustments for this example: different embodiments store the numbers as probabilities and perform prior-to-posterior conversion directly on the probabilities. (The numbers in this example are illustrative only.) The first box 500 shows the background rate 505 of a bloody nose and pain complaint, such as derived from the P&I system: for this patient illustrative example, his prior odds 505 are 1:4, and thus against. An X-ray was performed to look for fractures as shown in 510, and shows no fractures of the two nasal bones. The likelihood 511 that a negative result adjusts the prior in this example is ⅔, meaning that the X-ray is not a good test when it produces a negative as most broken noses occur on the border of the nasal bones to each other or the skull and thus fractures do not show up on X-rays: the negative result merely ruled out interior cracks of the nasal bones or cracks of the skull or surrounding areas. The next step performed was a palpation of the nose as illustrated in 520, which determined that the nasal bones are now misplaced. This results in a positive likelihood 521 of 30, thus pushing the odds greatly in favor of surgery. Closed surgery was performed as illustrated in 530, confirming the diagnosis and resulting in the odds ratio being set to +infinity, or probability of 1 (535, or as set by =1 in 531). There are multiple possible sources of likelihoods: query results from the P&I system based on incremental or cumulative queries of the state known at the time of each step on the path (a cumulative query may not produce a result, even if incremental queries do, and thus incremental may be performed as a backup to a failed cumulative query); direct input from a knowledge repository such as based on research (as many procedures are reported with their likelihoods or sensitivities and specificities); historical analysis of the practice and patient sets, including patient specific knowledge for recurring or chronic conditions; or overrides or input from the physicians themselves into the patient record (such as an accepted override).

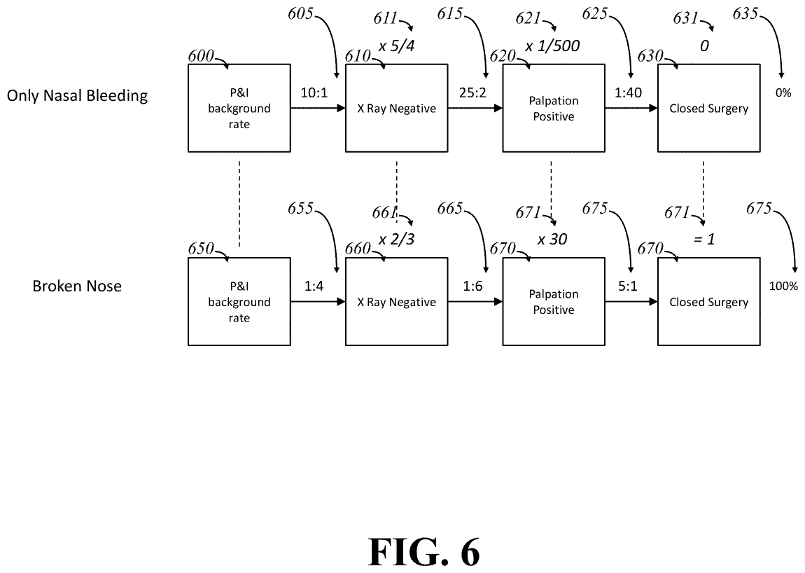

Note that the patient path may be represented in the form of a directed graph. In some embodiments, the path is stored and processed as a graph. This creates multiple possible “actual paths” through the graph. One possible advantage of this is that it allows for competing or overlapping but distinct diagnoses to exist; another is that it can represent uncertainty. Furthermore, the branching possibilities become important when revisiting hypotheticals, and in extending the patient path into the future, where multiple branches exist within the model based on outcomes of processes that might only be described statistically, such as with decision state space exploration, described below. Nothing in the term “patient path” should be used to require that the path be a linear chain: the term path is borrowed from the notion of the path integral in quantum physics, which is really a way of representing all possible state transitions. In the simplest case, such as shown in FIG. 6, the multiple graphs are parallel but non-intersecting, and represent different hypotheses chains. Each actual action that occurs is appended to the tail of each chain, thus producing multiple competing adjusted odds or probabilities (600-635 is one chain, 650-675 is the other). In these embodiments, the likelihood adjustments may be different based on the different suspected diagnoses. In the example, the hypothesis of “Only Nasal Bleeding” 600-635 has different L+/L− adjustments than that of “Broken Nose” 650-675, as the path leads towards confirmation of one and rejection of the other. The chains need not be merely dedicated to diagnosis. There are other ways of storing these effects. In another embodiment, the prior probabilities are stored as proper distributions rather than just as one number. A full distribution may be stored as a representation of the discrete or continuous priors and posteriors. The likelihoods become distributions too, aligned in the “x” axis along different beliefs. The quotes for “x” axis are because beliefs may not be dimensionally distributable in some embodiments. These different embodiments can, of course, store the same information: the parallel chains are merely labeled with the belief and are thus vertically stacked and merged into a distribution representation. However, some embodiments employ both: this is useful when some beliefs are in fact representable by a continuous or dimensionally-representable quantity while others are discrete. (The task of assigning representations is similar to the task of input and output coding in neural networks, and the art around representations is well understood.) Further embodiments generalize the structure to that of a Markov model (simple or hierarchical). One such embodiment uses the Markov model with each node representing the belief (or a non-overlapping decomposition of the belief space), with the action vectors constructed from the nodes of the patient path. A further embodiment disposes of the Markov property, and uses approximation methods to resolve the possible solutions.

Some embodiments associate with each belief not just a probability but an attendant uncertainty. One embodiment represents the uncertainty through confidence intervals, and updates the confidence intervals using associated confidence of the likelihood adjustments. Another embodiment represents the uncertainty through dependent attribute specific uncertainty, such as dependent attribute confidence intervals. This changes one confidence interval in the previous embodiment to a dependency list of confidence intervals, and applies the uncertainty of each step to produce a compound uncertainty. Further embodiments represent the likelihood of each step as also possessing uncertainty. One such embodiment is to represent likelihood at each belief as having a confidence interval. Other embodiments build on the above embodiment by representing continuous confidences rather than merely cutoff intervals. One further embodiment uses estimation statistics.

Note that the type of inferencing used in the development of the risk profile is a choice of embodiment. Some embodiments employ only a frequentist-based use of Bayes' theorem, and no not represent beliefs but rather frequencies. In those cases, a null input set may be represented by no frequencies rather than uniform belief. In other embodiments, the inferencing may occur based on maximum indifference rather than on belief. These are epistemic questions, and the point of the disclosed invention is to process the data based on the assembler's or operator's choice of embodiment corresponding to their desired epistemic belief.

The resulting risk profile is, in some embodiments, a dynamic data structure representing not just the current range of possible patient conditions (often hidden variables in practice) and the uncertainty in the data gathering and cohort identification, but the possible changes to the range based on hypothetical adjustments to the past or future of the patient.

One possible advantage of the risk profile is that it does not collapse the uncertainties into an artificially certain diagnosis/treatment path, but retains the uncertainty and multiplicity of diagnoses and treatments.

Simulation and Decision Engine and Betting Charts

Both Bayesian inference and statistical estimation (such as p-value hypothesis testing) have flaws. Pure Bayesian inference makes unrealistic assumptions about the beliefs, and buries those assumptions in the processing. This garbage-in-garbage-out property can, when coupled with a misunderstanding of statistics, lead to a misunderstanding of the decision state and likelihoods. Statistical estimation also suffers from the problem of misunderstanding of statistics: a p-value is itself a random variable, and moreover the conclusion that a sufficiently low p value means the hypothesis is true is not necessarily current; rather, it is the opposite of the proper conclusion, which is merely that a random event will masquerade as well as the given set of data. (p-value tells the probability of current data given the null, not the probability of the null given the data.) Sometimes the base case that is produced is noisy—but how the case changes with the assumptions can still reveal tremendous insight.

This motivates the need for the Simulation and Decision Engine in some embodiments. Decision engines are certainly not unheard of in the practice of medicine: they form the basis of genetic counseling, for example. But these are usually determinative and static from the point of view of the practitioner: the inputs are taken and an output derived, with little insight to the practitioner of why the number was arrived at, the dependencies, and the sensitivities to the dependencies. For example, it is diagnostically relevant whether the resulting odds are attached to properties measured of the patient or whether they are population specific only. As mentioned above, this is necessary to ensure that the different cohorts with different patient paths do not get treated alike. Another example of this: it is well understood that obesity increases the risk of type II diabetes. However, that risk isn't uniform to each patient—and this forms a complex epistemic cycle where confirmation bias can be baked into the behavior. As an overly simplistic example: if using an epistemic prediction engine, and the only information entered in is that the patient is morbidly obese, it will rather confidently announce a high odds of type II diabetes. One test for fasting glucose that shows no diabetes will downweigh the odds but will not set it to as low as for someone who is thin, even though diabetes can be defined as high fasting glucose. Of course, further tests with low fasting glucose will drop the odds further, but they may not be ordered because confirmation bias sets in. What got missed is that some people do not develop metabolic syndrome from obesity, and that the correlations have an unknown uncertainty. If the physician had the ability to temporarily suppress the weight inference, however, she could see the change in the odds based on the tests and then use her judgement to decide how much to weigh the prior belief.

A separate problem is that medicine is not practiced with a notion of total risk, but only of risk of disease. Total risk is a measure of possible interventions that assigns not only the risk of pain, suffering, complications, or death of having a disease but also of the treatment. Closely related is total utility or total cost. This too is an artifact of the collapsed diagnose/treat cycle. In an outpatient setting, typically physicians push for a specific diagnosis that they feel is most likely, and then focus their treatment regimen on that. In doing so, they have disregarded the possibility that they are wrong and thus that there is an opportunity cost. They have also lost track of the side effects of the treatments they are ordering. This is why most patients who present with lower back pain get offered low-outcome, high-pain surgery. Each individual step of the way is locally reasonable (if not locally maximal), but the outcome is not even close to optimal. Emergency medicine tends to be different. In an emergency department setting, physicians often do not have time to perform precise diagnostics, and therefore have to blindly treat, being conscious of the risks of being wrong and the harm they may cause. A simplistic example: broad-spectrum antibiotics, coupled with corticosteroids, for treatment of pneumonia-like breathing difficulty. The steroids can cause a bacterial infection to get worse; the antibiotics do not treat idiopathic bronchitis. Together, they treat most patients well with lower cost and risk.

Thus, the Simulation and Decision Engine, which in most embodiments acts as a probability playground for the physician to perform what-if historical and future analysis, to see what could be the way to go, and to glean a better understanding of why the system presents different diagnostic and treatment possibilities.

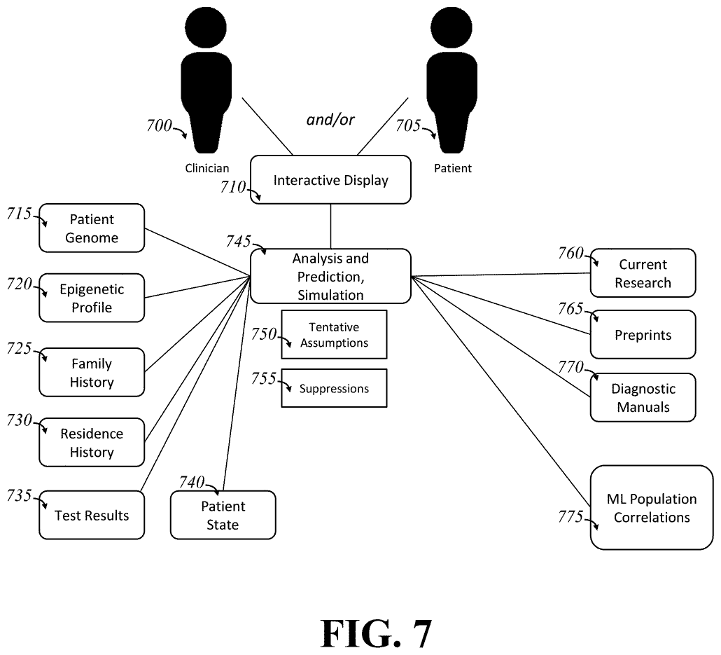

FIG. 7 shows inputs and outputs into the simulation and decision engine in some embodiments. In many embodiments, these inputs are derived through the risk profile, which is analyzed and adjusted by the simulation engine. Data sources may include validated medical research and early indications of research 760, preprints 765, diagnostic manuals 770, prior correlations such as those derived from machine learning 775, and also what were the greatest factors and by how much in prior correlations such as population, patient origin and family, 725 employment, activities, residence history 730, genome 715 and epigenetic profile 720, and patient state 740. Through an interactive display 710, the user (which may be a clinician 700, or a patient 705—unsupervised or supervised by the clinician—or anyone else for that matter) is able to see and navigate the decisions that went into the process.

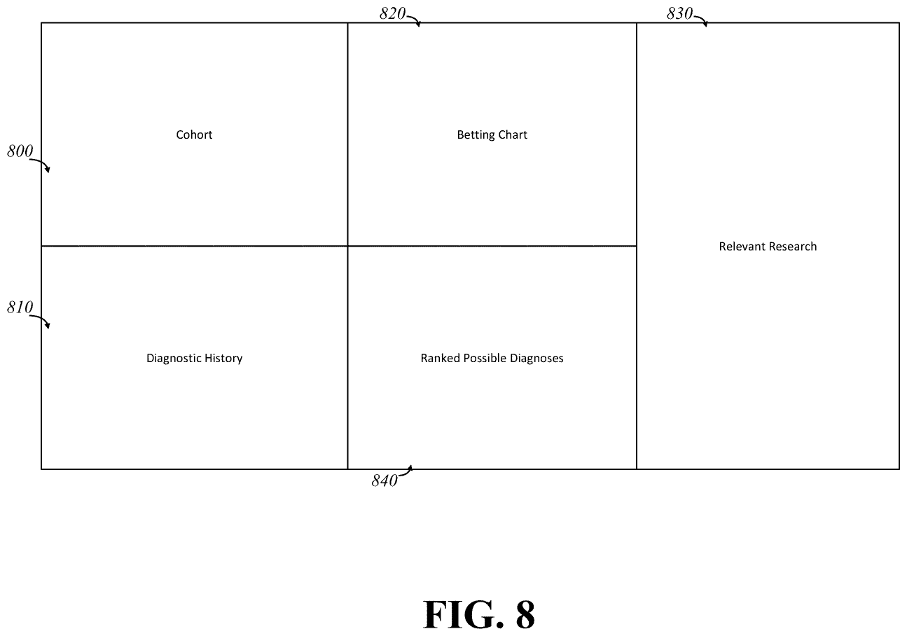

The various information sources and reasons are pulled together for display. Some embodiments have a display such as shown in FIG. 8. A likely cohort 800 that the patient belongs to is displayed. In one embodiment, the cohort is displayed as a text list of the attributes: a further embodiment shows them in the format of the Table 1 digestion table. In another embodiment, a graphical avatar of the patient is shown, assembled from the representative cohort; in a further embodiment, prior injuries are also shown when medically relevant to the suspected diagnoses. A diagnostic history 810 is displayed. In some embodiments, this is the patient path or a relevant subset. In some embodiments, the diagnostic history is displayed as a workflow. Between the cohort and the diagnostic history, the user in some embodiments is able manipulate the display to add, adjust, or suppress individual data items. In some embodiments, the modification is highlighted in color, shape, texture, size, font, or other visually distinctive method to allow the user to see what changes have been made. In this way, a user can determine how the results change based on the various inputs. In some embodiments, the system produces individual or joint sensitivities of each data item and displays that, thus using automation to avoid having the user have to manually derive the sensitivity by playing with the data item. An example would be for a risk analysis of type II diabetes, where the weight of the patient would be shown as highly sensitive. One embodiment determines this by dependency propagation through the risk profile; another uses numerical randomization methods to run multiple adjustments and reduce the sensitivity without regard for the specific methods of the risk profile's construction being necessarily known.

Possible diagnoses 840 are displayed. In some embodiments, these are ranked from most to least likely given the condition. In some embodiments, when data items are changed, the change from the old to new rank is shown. In some embodiments, the diagnoses are shown in a heatmap, a visual tool that changes the color of a region representing the diagnosis based on its likelihood or relevant factor. In some embodiments, diagnoses are replaced with beliefs in general. In some embodiments, related beliefs are displayed with their relationships, such as shown in closer proximity, or attached with arrows, lines, connectors, or other visual markers. In some embodiments, overlapping relatable beliefs are shown in a space-filling diagram, such as a Venn diagram, that allows for the overlaps to be shown together.

Relevant research 830 is displayed in some embodiments. In one embodiment, the relevant research is compiled directly from a search of a database based on data items on screen that produce a match. In another embodiment, the research is recovered from data sources such the P&I system, which then allows the physician to see what reasoning the system employed. In a further embodiment, the relevant research is highlighted or otherwise labeled to explicitly show how much weight it was given by the system. In another embodiment, the relevant research items are also shown with the derived inferences (such as the digester tables Table 2).

Betting charts 820 are shown in some embodiments. A betting chart, as used here, is a presentation tool to let the user understand (and in some embodiments, manipulate) the risk calculations for the options going forward. One key in some embodiments is to explicitly decouple the certainties of the diagnoses and the treatment options, as presented in the betting charts. An example: if the patient presents with symptoms fairly representative of pneumonia, but no tests have been performed yet, the diagnoses presented may be shown covering bacterial infection, viral, allergy, or idiopathic, all with low confidence (and in a Bayesian model, with rather equal beliefs at the low confidence) but all clearly above non-fitting or irrelevant diagnoses (such as a dislocated shoulder), which in most embodiments are not produced or not displayed. However, the betting chart may present a combination proposed intervention: treatment with antibiotics proper for pneumonia, a corticosteroid, and an antihistamine, and return for monitoring in a few days. This joint intervention proposal may then be displayed with high confidence—certainly higher than any of the individual diagnoses, but collectively containing the joint probability of all three of them, as the total diagnosis space for the given condition is sufficiently covered by the three diagnoses and their treatments. In other words, the point of the betting chart is to roll up all of the individual uncertainties of the diagnoses the system is holding (based on patient paths, usually), and presenting a range of reasonable actions. Furthermore, the betting charts are intended in some embodiments to also show the various risks of performing the procedures. Procedure risk may be captured in different ways, such as by the risk of failure of the procedure, the costs of the procedure (especially to the patient), the side effects of the procedure, the future risks of procedures needed in the future based on the decisions possibly made today (including the null decision). The particular form of the risk can be selected and modified by the user in some embodiments.

Betting charts need not merely be focused on future treatment proposals. A betting chart can be produced historically, to highlight the various changes to the likelihood space based on specific steps in the history of the patient as well. This is useful for capturing what influence a diagnostic step or test or intervention had on the system's assessment of the patient.

Another way to think of betting charts is that in some embodiments it captures the likelihood space produced by the inference engine and gives the user direct control and influence over how decisions are produced. Traditionally, a decision engine (also known as a decision support system) directly coupled to an inference engine: the system determines the likely best decisions to make based on the facts in hand. In doing so, it is usually rather opaque, and certainly not well designed to allow human physicians to be a part of the diagnostic and treatment process. Often, in fact, the stated goal of clinical decision engines is to replace physicians. Although one could employ the present invention to do just that, the point of most of the embodiments of this invention is to overcome that hurdle and place the physician back into the driver's seat, and then to extend it to include the patient as well, thus removing the opacity of traditional clinical decision engines. The state of the art is fairly limited in traditional decision engines because of this: they are used in narrow cases, such as radiotherapy where determining the appropriate angles for radio knife procedures, or for trauma evaluation where they are able to help assess for an emergency call center what level of crisis response could be needed. Beyond that, most of the use cases of the art are futures, and the proper, functional integration of clinical decision engines into general medical practice has been elusive. Many of the embodiments of the present disclosed intervention is able to achieve that elusive goal, in large part, because they do introduce the physician properly into the workflow and allow the users to see why a system is “thinking” what it is and what to do about it. In some embodiments, the user is able to directly change the specific bet that is being queried.

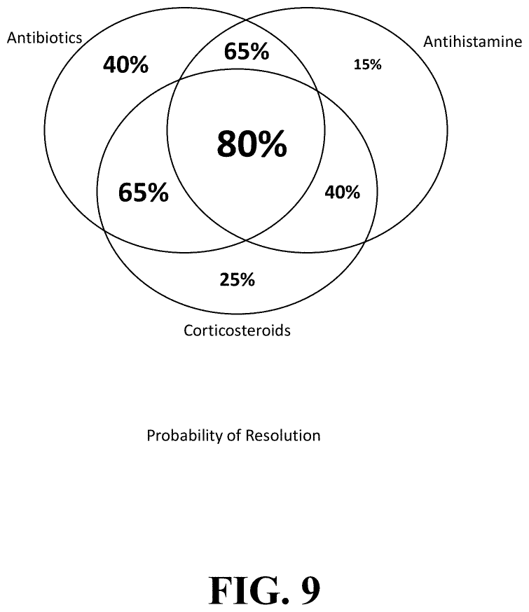

One example of an embodiment of a betting chart is shown in FIG. 9. This is a simplistic version, meant to teach the underlying properties of the disclosed invention. The user has, in this example, selected the bet query for total probability of resolution of lung pain and difficulty breathing. (Again, the numbers are for illustrative purposes only.) In this simplistic example, the probabilities of resolution for antibiotics, antihistamine, and corticosteroids are shown, as derived from the risk profile and the inference engine. In some embodiments, the reasonable or likely possible actions (or beliefs if the betting chart is placed into historical mode) are shown. In a further embodiment, options are shown in an area filling model. The above example is produced by an embodiment that produces a Venn diagram, and further shows the numerical probabilities emphasizing greater odds with larger text. The joint probabilities for overlapped treatments are shown. Other embodiments produce text, showing the joint or relational aspects of the options in words, numbers, or symbols. The above example tends to support traditional waterfall medicine, where first a diagnosis is made and then a treatment is performed.

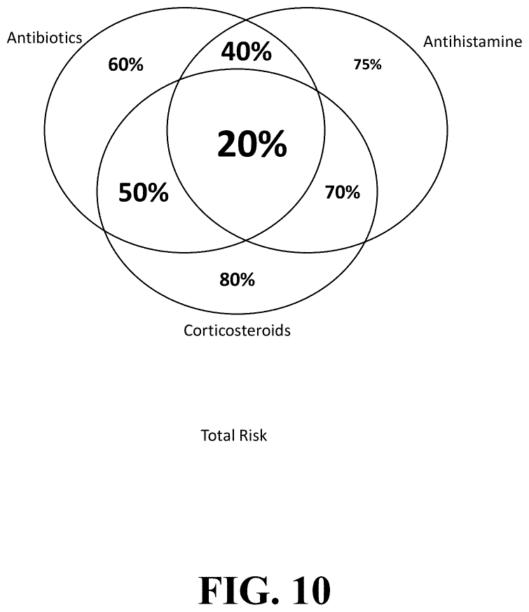

More interesting is when the user switches the mode to total risk, producing a simplified example in FIG. 10 (again, the numbers are for illustrative purposes). Risk can be evaluated on a number of different scales. In this example, the scale is from 0-100%, where 100% is a measure of ongoing suffering of the patient. Other embodiments use different measures of risk, such as risk of pain levels being above a certain threshold, risk of death, risk of cost, risk of inconvenience: these are all encapsulated in the Risk Model, described below. When looking at this example betting chart, the lowest risk is clearly shown to be in the joint treatment. Notice how the total risk is not one minus the probability. Taking corticosteroids alone is quite risky, even if the diagnosis model highly suspected idiopathic inflammation, because if there is a bacterial infection, even a slight one, the suppression of the immune system can encourage the bacterial infection and cause rebound pneumonia. Of course, this particular example is fairly simple, as physicians today already generally know to prescribe the combination to prevent this problem. However, physicians are not capable of remembering everything for all diseases, which is the point.

The betting chart shows the user where they should focus their efforts, and allows the user to shift from overemphasizing diagnosis to emphasizing diagnosis and treatment options. Furthermore, because the sources of the treatment options come from the risk profiler and the decision state space explorer, the system automatically captures the possible next steps and gold standards/best practices of medicine. For example, the two above betting charts are produced without any testing. However, imagine a case where a simple blood test can eliminate the need to take the corticosteroid, for a patient who has diabetes and thus is extraordinarily affected by the side effects of the corticosteroid. If the blood test takes one day to turn around, say, the decision state space explorer can take the current patient path and risk profile and add future possibilities of the test being performed (+ and −) with delayed treatment versus treating today. These other options can be displayed as well, and may even, depending on the facts of the case, show that the test and delay option is the greater option. In some embodiments, the user can manipulate these various options and suppress them or alter them, to further the future timeline.

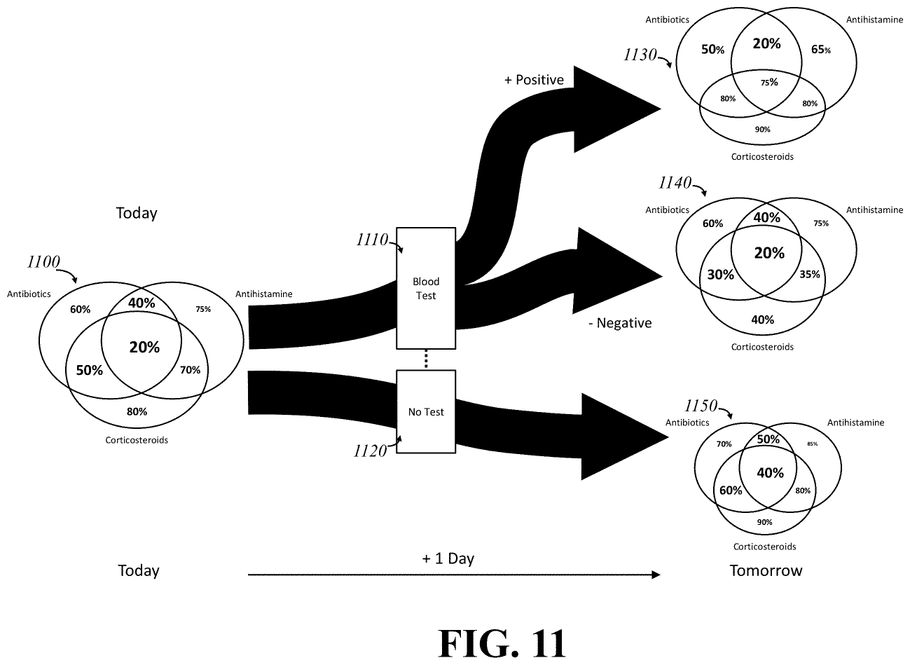

In some embodiments, the forking nature of the decision path, past and future, is shown. FIG. 11 shows such a display, based on the hypothetical blood test mentioned earlier from FIG. 9, shown as 1100. In this example, the forking possibilities of a not yet performed blood test—and the result's possible influences on the individual betting diagram—are shown. Not giving the test and waiting a day will increase the risk to the patient: thus, the risk numbers have gone up. This is the null case path through 1120. Giving the blood test (the path through 1110) will also increase the risk to the patient, because of that delay. However, a positive result (results 1130) will rule out the need for risky corticosteroids, and thus produces a different betting chart favoring not giving corticosteroids. A negative result fails to rule out steroids, and in this hypothetical case decreased the risk of giving steroids as shown in chart 1140. The particular example above has multiple risk numbers, but in some embodiments the risk numbers themselves are hidden, or displayed as a different risk rating (such as 1-10), or as risk thermometers such as with red being hot and green being cool. As usual, actual numbers are produced by patient specific analysis. In some embodiments, the decision paths that lead to a more favorable risk balance are highlighted, or otherwise distinguished. In some embodiments, the change to the risk is labeled or displayed along the paths. In some embodiments, the display is an adaptable layout of the timeline in the past and future, and allows the user to manipulate the range of times shown to go back in time and revisit decisions, or to go forward in time and see hypothetical situations. In some embodiments, the user can introduce state changes, to see what would have been or what would be: for example, a user could go forward in time and intercept a specific moment in time and introduce an injury, or a diagnosis, or other hypothetical.

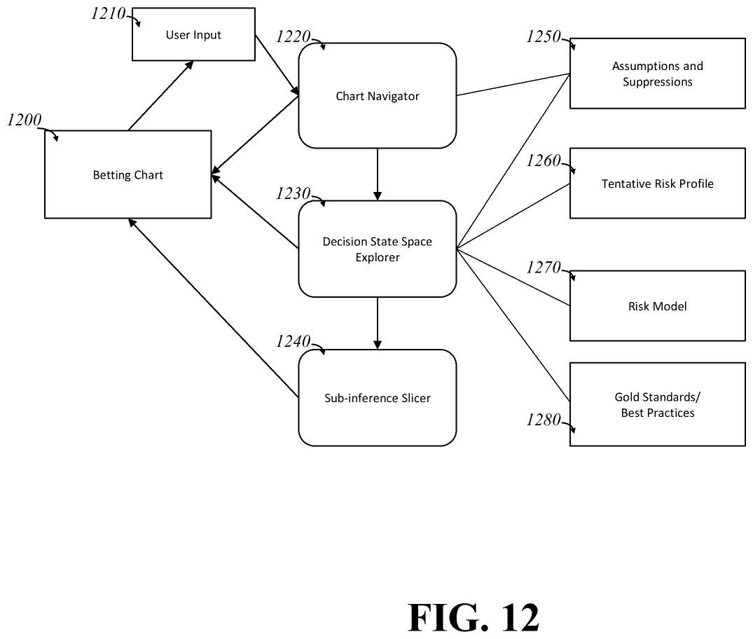

The betting charts are produced in some embodiments by the process shown in FIG. 12. As the user adjusts the timeline view or alters the query state of the engine, including assumptions and suppressions 1250 of fact or possibilities, a chart navigator 1220 recalculates and updates a chart 1200. If the chart is required to move into a different region of the state space, or if new parts of a set of charts are required that are not already represented, a Decision State Space Explorer 1230 is called into action. The decision state space explorer requests the production of tentative risk profiles 1260 and synthesizes that interventions, taking into account a risk model 1270 in use at the moment and gold standards/best practices interventions 1280. The risk model contains the type of risk that is being viewed. In the simplest case, the risk model requests merely the probabilities of resolution. In more complicated cases, the risk model can represent an arbitrary utility or loss function, but usually is constrained to reduce pain and suffering of the patient. In some embodiments, the risk model takes into account the patient's stated goals: for example, a knee injury that does not reduce working ability or a reasonable person standard quality of life but prevents long distance bicycle riding may start off with low impact to the risk model, but the patient can request that the model increase the weight on restoring the knee to full use. Some embodiments use a programmatic interface for expressing an arbitrary formula based on available data items to produce the particular utility or loss. In some embodiments, the decision state space explorer produces the set of decision possibilities by consulting the gold standards/best practices, where the gold standards are stored as a database of interventions and likelihood adjustments.

In further embodiments, the interventions and adjustments are stored as nodes with multiple inputs and outputs, in a way that can form a directed graph: in one embodiment, the interventions form the nodes and the various results form the edges, tagged with the likelihood/risk adjustments. Note here that in this case the form exactly matches that of the patient path. The patient path, as you may recall, can often be represented or interpreted as a chain, under the assumption that history has only one outcome. However, as it was originally described, it is actually the collapsed part of a probability graph, collapsed along the lines of how the history is remembered. Even a collapsed path can be re-expanded, showing the roads not taken—one simple method of doing so is to recover the corresponding intervention from an intervention database such as the gold standards/best practices database, and re-add the pared-down edges. In some embodiments, nodes and edges are not forced to be completed, and can be left with reference pointers for future computation, such as in the manner of generators or symbolic computation. In those embodiments, the edges or nodes can be followed upon need or request. In some embodiments, the explorer appends to the patient path all matching intervention nodes for future possibilities. In others, the database is sorted and only those entries that pass a certain filter of relevance (such as improving risk or matching the diagnosed condition) are produced and added. Because the process may be infinite if forced to completion, in some embodiments the explorer only explores as far as necessary to fulfil the needs of the display. The processing of the risk transitions in the explorer produces a kind of all-state path integration, in that the explorer may be used in a way to endeavor to find the more likely paths but to also take into account the less likely ones. In some embodiments, the assembly of the future possibilities is directed by the user via input 1210: in one embodiment the user manipulates a currently terminal part of the graph, and then selects what the next step should be. In a further embodiment, the user can select from a choice of proposed next steps, such as derived from the best practices database. This may have the advantage of guiding the user to the better options for intervention and not applying a nonsensical or unusual one (unless configured to allow overrides). In a further embodiment, this works through a palette of possibilities, and their affiliated relevance information such as the maximal risk reduction or median risk reduction. In some embodiments, the graph is assembled forward breadth first; in a further embodiment, only a subset of the high watermark is processed, based on its ability to influence the goal of the user (such as risk reduction). Other embodiments use other graph traversal techniques. One way that users can interact with these embodiments is to explore what the value of future possibilities would be, and to choose to foreclose them or amplify them, thus creating or modifying a suppression or assumption and allowing a partial or whole recomputation and redisplay of the betting charts and the risks.

A subinference slicer 1240—an optional piece in most embodiments but present in some embodiments—can be called into action to eliminate entire inferencing paths that the user determines are not of interest. In one embodiment, the elimination renormalizes the risks and probabilities for the discounted path—thus being different from merely hiding things in explicitly carving out part of the inference and decision space.

In some embodiments, the betting charts are presented to the patient, such as being printed out or emailed, or viewed on an app. This way, the patient can have something to look at if they are sleeping on a decision and need to weigh the consequences.

One possible advantage of using such a decision system is that the physician need not remember the appropriate gold standards or best practices for a given condition. Because many of the embodiments disclosed above automatically consult with best practices to assemble computationally a forward set of paths, with their risks and rewards highlighted. The physician can be reminded for example that for a broken nose an X-ray is unlikely to change the outcome, or that it will actually increase risk because of the time to heal.

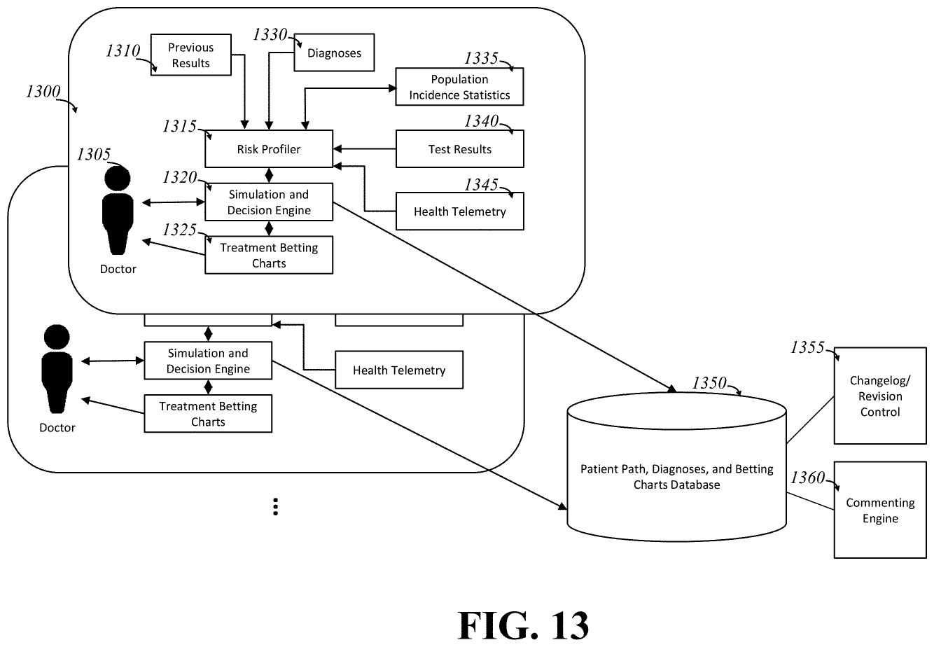

A further set of embodiments is to allow the collaboration of multiple physicians into the same simulation and decision engine. FIG. 13 shows some embodiments that have collaboration features added. 1300 illustrates the embodiments of FIG. 1. A database 1350 is provided to store the patient paths, diagnoses, and betting charts, including the assumptions and suppressions. Along with the database is a changelog/revision control system 1355, to allow each user to see what changes the user has made historically, and which changes other users have made historically. In some embodiments, there is only one current database entry, and everything else is historical. In other embodiments, each user can have one or more own copies of their “head” state, and use merging techniques such as known in revision control to compare differences and choose what changes to accept or not. Furthermore, in some embodiments there exists a commenting engine 1360, which allows a user to comment on a data item within the display and its underlying source, or to comment on a change. In an embodiment, the changes can be marked as proposed. In some embodiments, a full transaction control system is employed to allow the user to batch changes and chose to commit overwrite, merge, or rollback.

Why allow this collaboration? In some embodiments, specialist or experts for particular conditions or interventions review the decision space of a patient (past patient paths or simulations), and are allowed to suggest new simulations or alter existing ones, or to revisit past states and introduce second opinions and schedule repeated tests or interventions even if previous ones have been marked as conclusive. One advantage of this may be that it converts a linear stream-of-consciousness health record process into one that can be altered, revisited, proposed, and reunderstood without causing unusual disruption to the workflow or require deleting history. For example, a misdiagnosis that lead to a set of treatments is recorded forever in traditional EHRs with the diagnosis codes intact. A physician who discovers the error can go and remove the current diagnosis codes, but now the work that was done for the false diagnosis remains in the system unflagged. A future physician looking through the records might see the treatments and assume the diagnosis is still valid, and even restore the diagnosis codes. It's especially tragic because diagnostic codes are used for Medicare and other billing in traditional medicine. But with the changelog system recording exactly who, why, and when a path was curtailed or recreated, it becomes possible for the physician to investigate and come to the correct conclusion as to the state of the patient without any ambiguity.

The role of the physician-patient relationship is the heart of medicine, and tools, systems, and mechanisms designed to replace that relationship itself are unlikely to go down well with patients for humanistic and cultural reasons, even if such a technology could easily be created. A possible advantage of the invention disclosed herein is that it allows for the primacy, and the improvement of the quality of, that very human relationship. Even though it is a well-known fact that computers can, using straightforward and incomplete inference engines, diagnose many diseases better than a person on average, one possible advantage of systems such as that disclosed herein is to manage not the average but the extreme case, and a human may be of value culturally if not mechanistically in handling those situations and guiding them towards correctness. This motivates the various embodiments that allow for the user to adjust, play with, override, overrule, alter, and otherwise modify what the computer presents. To the extent that this can lead to a physician intentionally or otherwise introducing confirmation or other biases into the health record, some embodiments that record the changes can also compare the changes made and create a historical and modern record of what those changes are. For collaboration embodiments, the collaborators can review those changes. In some embodiments, those changes are flagged and ranked or processed into priorities for review by others: the selection can be entire or based on statistical sampling methods as understood for quality assurance. In this way, the quality standards of the physicians themselves, or the physician-patient dyad, can be tracked and explored for long term quality implications. Further embodiments can immediately flag changes classified beyond certain filter levels, such as risk changes, individual procedure risk, or amount of historical or current state has been changed, and flag those for automated quality review or specialist consult. In this way, biases can be tracked and managed, in ways that no present EHR, with its deterministic workflow, can.

Some embodiments use an additional method of display for the individual intervention, diagnostic, or treatment steps. These are fact boxes. The fact box is a graphical tool, designed to highlight to the physician that part of the statistical properties of the intervention that are often quite difficult for humans to grasp, even when they are statistical experts.

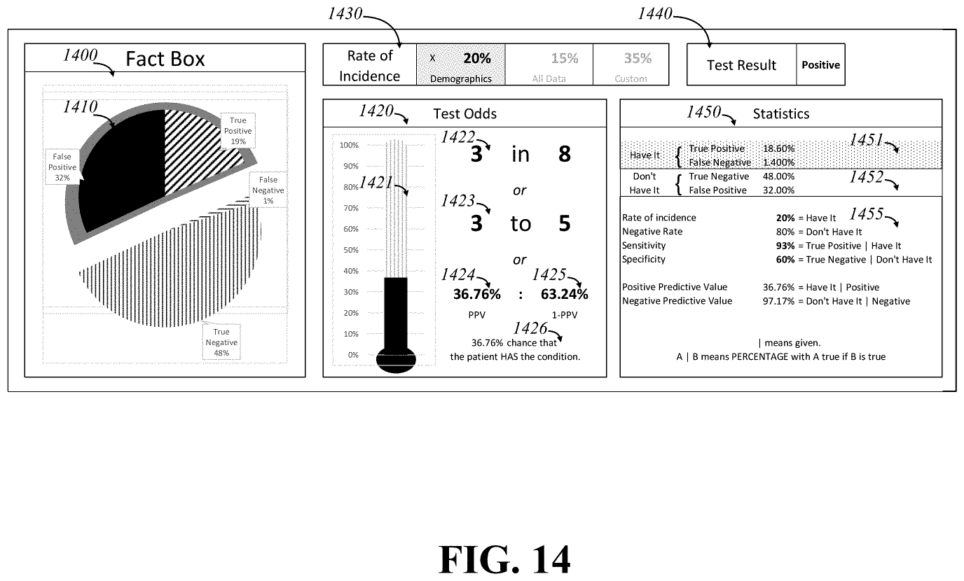

Fact boxes can be presented in the context of the patient and the treatment history, or out of context, with the inputs assumed or presented. FIG. 14 shows an example fact box for a diagnostic test performed with a sensitivity and specificity of 79% on an incidence rate of 6.7%. The false positive rate of this test is almost 20%. Unfortunately, people tend to forget what that means, and assume that a false positive rate of 20% means that a positive is a false positive 20% of the time and a true positive 80% of the time. However, that is not the case: rather, it means that in the case of not having the condition, a false positive is reported 20% of the time, and a true negative 80% of the time. This one error in understanding alone has led to anguish and false diagnoses and treatments in modern medicine. Any attempt to solve it by highlighting what is wrong is a value.

In some embodiments, the fact box displays, for the statistical power and known or mooted situation, the relative proportions of the population in question who thus have the condition or not. For the figured fact box, the display shows, separated out of a pie chart 1410, the population who have a positive result and wants to know what to make of it. In this example, the false positives greatly outweigh the true positives. In fact, the real power of the test can easily be seen not in the positives but in the negatives: in a negative result, the pie chart suggests that the predictive power is extremely high. Therefore, the test serves not as a good screening test for the disease but as a good screening elimination test against the disease: a positive doesn't mean much, but a negative is highly suggestive of a disease-negative state. In some embodiments, the information is conveyed graphically; in others, textually.

In some embodiments, the test odds 1420 are also displayed. Based on the test result and the patient cohort, the odds ratio 1423 and the probability ratio 1422 are displayed. In the particular embodiment exemplified above, there is also a graphical display of a thermometer 1424, showing how predictive the particular result is. In some embodiments, the detailed statistical and mathematical breakdown 1450 is shown, which may further provide a foundational understanding to the clinician or patient.

The value of such a graphical and visceral method is pretty clear. The above graphic is illustrative but the point remains: this particular diagnostic test is used often by a particular specialty as the first and often only diagnostic step before providing treatment. If the physicians performing this test saw a fact box—much the way that smokers see a surgeon general's warning or an eater sees the nutrition facts and calorie count—they are much more likely to be able to make better decisions about the value of the information they received.

In some embodiments, the fact boxes are available from the simulation display. In one embodiment, the fact boxes can be popped out and displayed over the top of or to the side of the main fact or screen element. In another, they can be printed. Note that the information contained in the fact boxes may be available directly from the P&I and best practices systems, and thus is another way to show why the risks or propensities change the way they do.

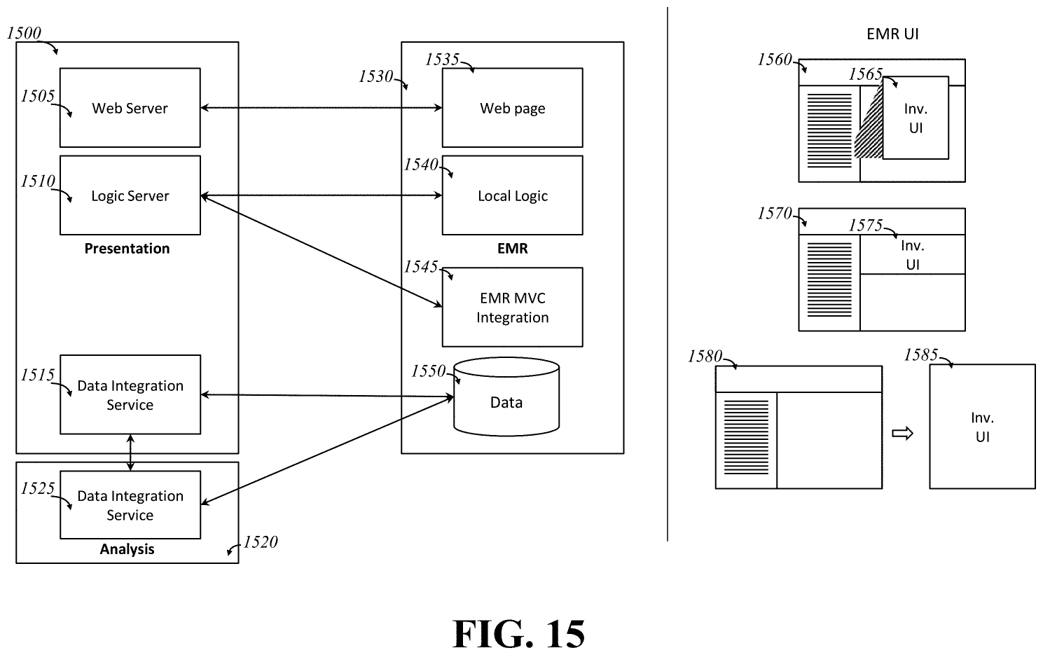

The disclosed invention may be employed as a substitute to a traditional EMR/EHR or as an adjunct. Traditional EMR integration is shown in FIG. 15. To allow for this sort of integration, some embodiments embed the simulator display in a web page 1535, driven by a logic server 1510 that can connect to the EMR MVC through integration 1545. Furthermore, data to populate the patient path is derived from the Data Integration Services 1515 and 1525 of both the presentation block 1500, which acts as the interface of the simulator display, and the analysis block 1520, which performs the creation of patient paths in the specific and P&I data in the general in these embodiments.

This disclosure requires familiarity with the state of the art in machine learning, statistical inference, and medical diagnosis. Terms like “detect” and “infer” are not necessarily absolutes, but may also refer to the increase in a determined value (such as likelihood or probability) or an increase in its confidence. Medical facts, statistical examples, numbers, and the like are for the purposes only of explaining the invention and its operation, and are merely illustrative.

It is the intent in this disclosure to teach not only the pure technological methods but the specific applications to various diseases and conditions.

In the description herein, one or more embodiments of the invention are described, with process steps and functional interactions. Those skilled in the art would realize, after perusal of this application, that embodiments of the invention might be implemented using a variety of other techniques not specifically described, without undue experimentation or further invention, and that such other techniques would be within the scope and spirit of the invention. The use of the words “can” or “may” in regards to the structure and operation of embodiments is to be construed as referring to further embodiments and configuration options, and does not require further experimentation or invention.

The scope and spirit of the invention is not limited to specific examples disclosed therein, but is intended to include the most general concepts embodied by these and other terms.

Although the invention has been described with reference to several exemplary embodiments, it is understood that such descriptions and illustrations are not limiting. Changes may be made within the purview of the appended claims, as presently stated, without departing from the scope and spirit of the invention in its aspects. Although the invention has been described with reference to particular means, materials, machines, and embodiments, the invention is not intended to be limited to the particulars disclosed; rather, the invention extends to all functionally equivalent structures, methods, machines, and uses such as are within the scope of the invention and claims.

I claim:

1. A method for electronically guiding the analysis and interpretation of medical information comprising steps of:
a. collecting statistics of propensities and incidences within a population;
b. collecting at least one of: test results and current diagnoses of a patient;
c. simulating for a user expected possible outcomes of changes in the medical state of said patient; and
d. displaying said simulation to said user
whereby said user may be able to gather a greater understanding of at least one of: the statistical possibilities of the effects of further testing on the significance of diagnoses; and the risk to the patient of treatment decisions.

2. The method of claim 1 comprising further step of: producing a risk profile of the patient.

3. The method of claim 2 wherein the risk profile is based off of the analysis of patient statistics using a patient path model.

4. The method of claim 1 comprising further step of: generating treatment betting tables for display to the user.

5. The method of claim 1 wherein said propensities and incidences is at least partially created by digesting to extract statistical rules from at least one of—published research, preprints, diagnostic manuals, gold standards, and best practices—using at least one of: automated entry, and manual entry.

6. The method of claim 1 wherein said propensities and incidences is at least partially created by extracting actual outcomes from the records of patients and identifying relevant cohorts of said patients.

7. The method of claim 1 wherein said simulation uses a navigator for exploring the decision state space of the patient, said decision state space populated at least in part using at least one of: patient information, assumptions and suppressions for the patient, a tentative risk profile, a risk model, gold standards, and best practices.

8. The method of claim 1 wherein multiple users collaborate electronically using a simulation and decision engine.

9. The method of claim 1 wherein the simulation and decision engine's user interface is integrated into that of an electronic health record.

10. The method of claim 1 wherein the user is displayed the statistical likelihoods of the patient having a condition, populated at least in part by at least one of patient history and prevalence and incidence rates; whereby the display of facts may ground the user in the true statistical nature of the patient and the historical evidence surrounding the patient and may avoid common misunderstandings of statistical inference, cognitive biases, and lack of complete understanding of the record.

11. An electronic system for guiding the analysis and interpretation of medical information comprising:
a. a collection of statistics of propensities and incidences within a population;
b. a collection of at least one of: test results and current diagnoses of a patient;
c. a simulator whose simulations provide user expected possible outcomes of changes in the medical state of said patient; and
d. a display for said simulations;
whereby said user may be able to gather a greater understanding of at least one of: the statistical possibilities of the effects of further testing on the significance of diagnoses; and the risk to the patient of treatment decisions.

12. The system of claim 11 comprising further: a patient risk profiler which provides a risk profile.

13. The system of claim 12 wherein the risk profile is based off of the analysis of patient statistics using an electronic patient path model.

14. The system of claim 11 wherein treatment betting tables are generated and displayed to the user.

15. The system of claim 11 wherein said propensities and incidences is at least partially created by digesting to extract statistical rules from at least one of—published research, preprints, diagnostic manuals, gold standards, and best practices—using at least one of: automated entry, and manual entry.

16. The system of claim 11 wherein said propensities and incidences is at least partially created by extracting actual outcomes from the records of patients and identifying relevant cohorts of said patients.

17. The system of claim 11 wherein said simulation uses a navigator for exploring the decision state space of the patient, said decision state space populated at least in part using at least one of: patient information, assumptions and suppressions for the patient, a tentative risk profile, a risk model, gold standards, and best practices.

18. The system of claim 11 wherein multiple users collaborate electronically using a simulation and decision engine.

19. The system of claim 11 wherein the simulation and decision engine's user interface is integrated into that of an electronic health record.

20. The system of claim 11 wherein the user is displayed the statistical likelihoods of the patient having a condition, populated at least in part by at least one of patient history and prevalence and incidence rates; whereby the display of facts may ground the user in the true statistical nature of the patient and the historical evidence surrounding the patient and may avoid common misunderstandings of statistical inference, cognitive biases, and lack of complete understanding of the record.
